(19) United States
(12) Patent Application Publication
(10) Pub. No.: US 2007/0039622 A1
(43) Pub. Date: Feb. 22, 2007

(54) Kufbag restraining device

(76) Inventors: John Thomas Foster, Westerville, OH (US); Glenda Louise Hill-Foster, Westerville, OH (US)

Correspondence Address: Davis & Kendall, PC, Suite 528, 27 N. Wacker Drive, Chicago, IL, 60606, US

(21) Appl. No.: 11/401,813

(22) Filed: Apr. 10, 2006

Related U.S. Application Data
(63) Continuation-in-part of application No. 10/952,586, filed on Sep. 26, 2004, now abandoned.

(51) Int. Cl. A61F 5/37; A61B 19/00

(52) U.S. Cl. 128/878; 128/869

(57) ABSTRACT

The invention provides a method and apparatus for prisoner restraint using a durable material having a front and rear side forming a bag. Attached to the front side of the material is webbing using a hook and loop velcro system. Grommet holes deposed in and through the material allow for plastic ties to pass through and secure the hands. A two-part buckle attaches to the webbing with one part affixed to the webbing and the other part affixed to the front side of the durable material preventing the use of the prisoner's hands for escape.

### CROSS REFERENCE TO RELATED APPLICATIONS

This application is a continuation in part of previously filed patent application Ser. No. 10/952,586 filed on Sep. 26, 2004.

### FIELD OF THE INVENTION

The invention relates to a method and apparatus for restraining and transporting a prisoner. The invention provides a means for safely securing the hands of a prisoner, without the additional use of bulky and cumbersome metal restraints.

### BACKGROUND OF THE INVENTION

The transporting of prisoners or patients has always presented a problem to security and police officers. Handcuffs and plastic cuffs only minimize the mobility of a prisoner's hands. Whether the hands are cuffed in the front or behind the back, the hands are not completely secured. A number of ways have been developed for defeating or minimizing the effective use of handcuffs, once attached to a suspect, due in part to the linkage between the bracelets. For example, in those instances where a person has the handcuffs attached behind their back, the present interconnection between the bracelet sections is sufficiently flexible to permit a cuffed individual to “step through” the handcuffs by pulling the handcuffs behind from their legs, which can be accomplished, for example, when the detained individual is seated in a police vehicle. Once the handcuffs are in front of the individual, it is much easier for the detained individual to run or to achieve better balance. Moreover, the individual would also be able to better access a shirt pocket, for example, to retrieve a hidden handcuff key, to obtain a weapon or to hide evidence. Additionally, the prisoners to engage in escape attempts by grabbing security officer's weapons. Wrist chains have been used to either choke or incapacitate the officers.

Standard handcuffs, such as those that are described in U.S. Pat. No. 6,311,529, include a pair of lockable cuff or bracelet sections, each of the bracelet sections being defined by respective accurate cheek and jaw members that are pivotally secured by means of a locking mechanism that is carried on the cheek member. The locking mechanism includes one or more gears that are engageable with a row of teeth carried by the pivotal jaw member, wherein the locking mechanism can be selectively disengaged and the bracelet sections opened by means of a key.

The present invention overcomes the limitations of the restraints as described above. The 4 Kufbag is a strait jacket for the hands. It can be used in conjunction with waist chains and handcuffs thus preventing the prisoner from having a free range of motion of the hands. The Kufbag covers the prisoner's hands and restraining device preventing the palms and fingers of the prisoner access or use.

### SUMMARY OF THE INVENTION

The present invention is a strait jacket for the hands. The invention uses durable material along with Velcro straps, handcuffs and plastic ties to cover and restrain the prisoner's hands in either the front or behind the back. The durable material covers the hands of the prisoner, with a pouch having left and right compartments.

The invention secures the hands together eliminating grasping. The hands are departmentalized keeping them from working in concert with one another, and a velcro alert system is installed to alert security officers if the prisoner attempts to escape.

The present invention comprises a durable material having front and rear sides. Attached to the front side of the material is webbing using a hook and loop velcro system. Grommet holes deposed in the material allow for plastic ties to pass through. A two-part buckle attaches to the webbing with one part affixed to the webbing and the other part affixed to the front side of the durable material. D-rings attached to the backside allow for waist chains to further restrict the prisoner's hands.

In operation, the prisoner's restrained hands are individually secured in the invention. The left hand is secured with the left side hook and loop strap. The right hand is secured using the right side hook and loop strap. The invention is closed around the prisoner's hands and plastic ties are fed through the grommets and fastened immobilizing each hand.

It is an object of the invention to provide a system for immobilizing the hands of a prisoner.

It is another object of the invention to further restrain the hands of a prisoner preventing the grasping or use of the palms.

Another object of the invention is to prevent the transmission of body fluids and infectious diseases.

### BRIEF DESCRIPTION OF THE DRAWINGS

The accompanying drawings, which are incorporated in and form part of the specification, illustrate an embodiment of the present invention and together with the description, serve to explain the principles of the invention.

In the drawings:

FIG. 1 shows a typical front view of the invention;

FIG. 2 shows the back view of the invention;

FIG. 3 shows a detail of the wrist straps; and

FIG. 4 shows another embodiment of the invention.

FIG. 5 shows a preferred embodiment of the invention.

FIG. 6 shows the invention in operation.

### DETAILED DESCRIPTION OF THE INVENTION

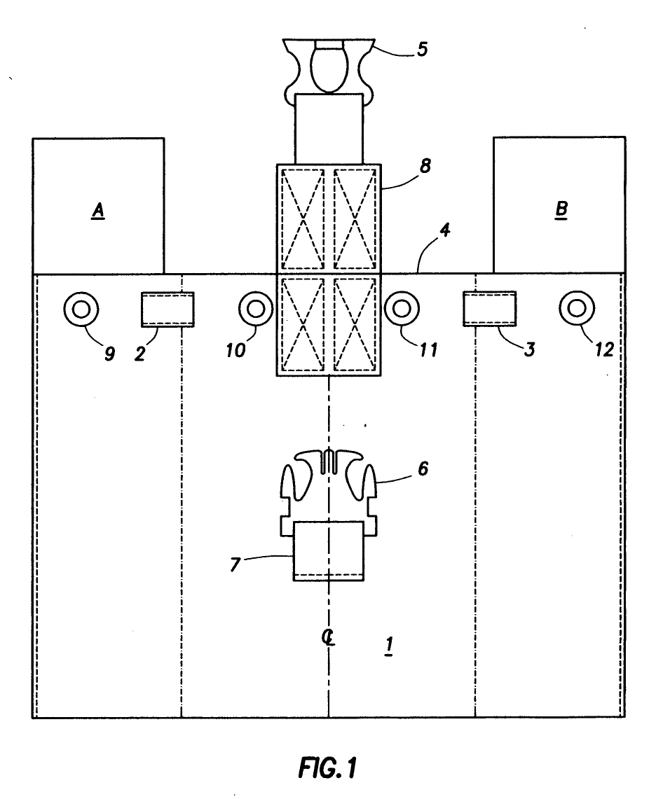

Referring to FIG. 1 the restraint device 1 is comprised of a sturdy fabric consisting either of canvas, nylon, or light plastic. In this embodiment webbing guides 2 and 3 are attached to the main body 4 to keep the plastic ties in place during the restraint process. Cap lock buckles 5 and 6 are attached to straps 7 and 8 that are affixed to the main body 4. Grommets 9-12 allow a rope, plastic ties, or string to pass through and allow for adjustment of the fit after a prisoner's hands have been restrained. Tabs A and B allow wrist straps to be attached to the restraint device 1.

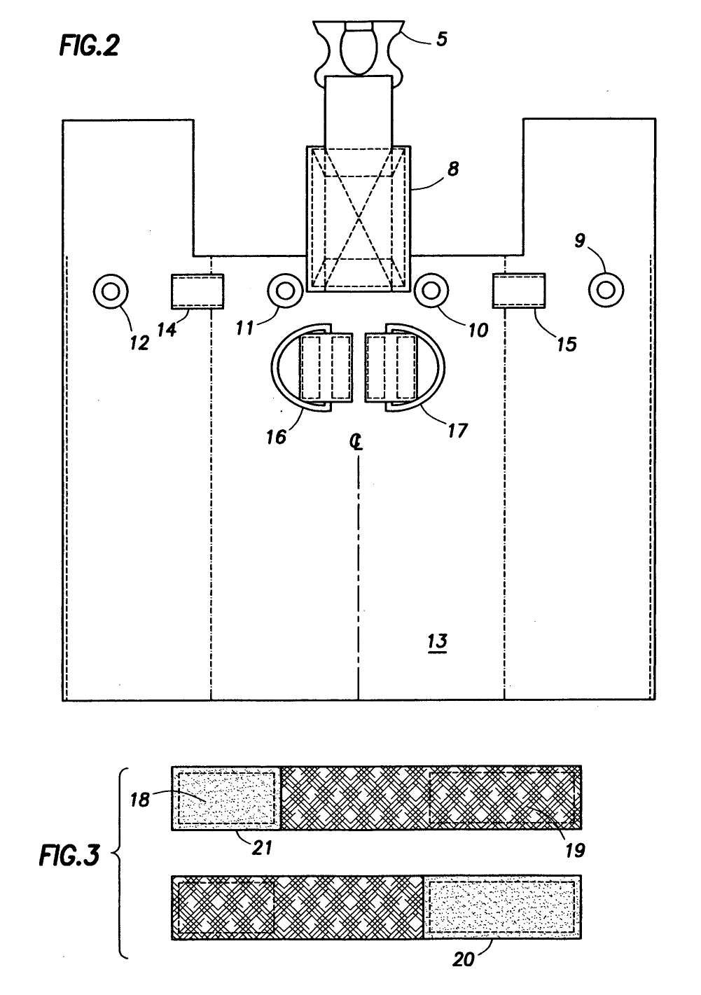

The backside of the invention 13 is shown in FIG. 2. Reinforcement webbing guides 14 and 15 are also affixed to the back body side 13. D-rings 16 and 17 allow the prisoner be chained in line with other prisoners or to a fixed position. In operation, the prisoner's hands are either restrained by conventional handcuffs or by plastic cuffs. Referring to FIG. 3, wrist straps 18 and 19 are attached to tabs A and B. Each have a velcro hook 20 and loop 21 system attached at each end of the wrist straps 18 and 19. The cap lock buckles 5 and 6 are interlocked with the prisoners hands inserted inside the main body 4 of the invention creating a bag or pouch. Either plastic ties or string is inserted in the grommets 9-12 to adjust the fit of the invention around the prisoner's hands. Thus, the hands of the prisoner are prevented from grabbing, reaching, or attempting escape.

### PREFERRED EMBODIMENT

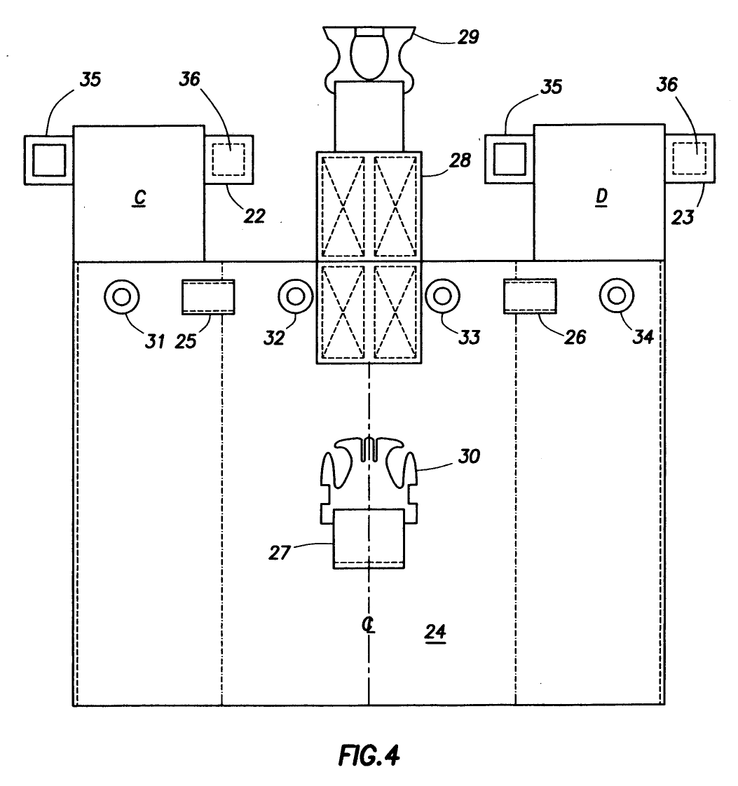

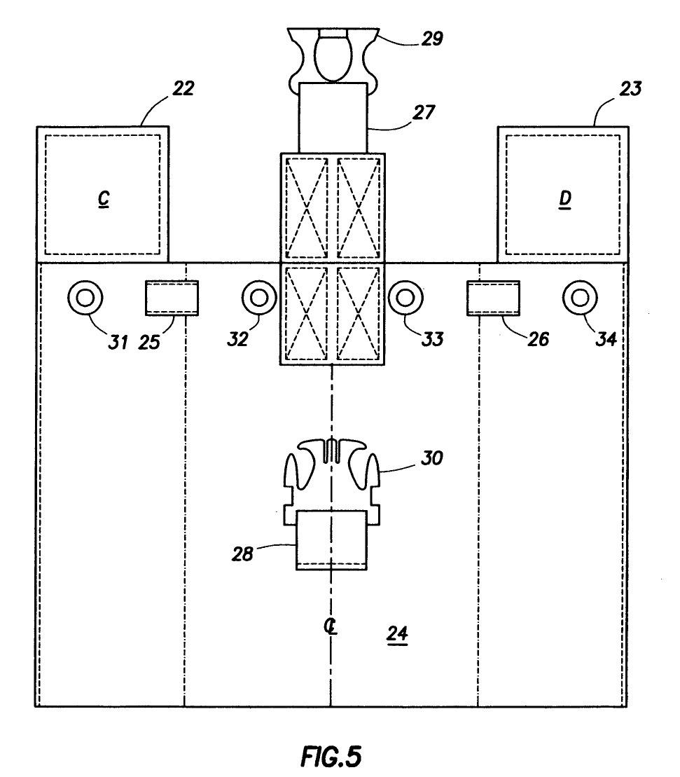

In the preferred embodiment the invention, as shown in FIGS. 4, and 5 tabs C and D have wrist straps 22 and 23 affixed. The main body 24 has webbing guides 25 and 26 attached to the front side of main body 24 to assist in holding the plastic ties in place during restraint. Straps 27 and 28 are attached to the main body 24 in such a way as to allow for cap lock buckles 29 and 30 to swing freely. The cap lock buckles 29 and 30 are designed so that each has a secure fit snap lock and release. The cap lock buckles 29 and 30 are positioned so that upon being snapped into a locked position, the main body 24 creates a bag around the hands of the prisoner. A sewn seam creates a left and right side compartments for the hands. Grommets holes 31-34 allow a string, plastic ties, or rope to be threaded through the invention body 24 to allow the user to adjust the invention around the hands of the prisoner.

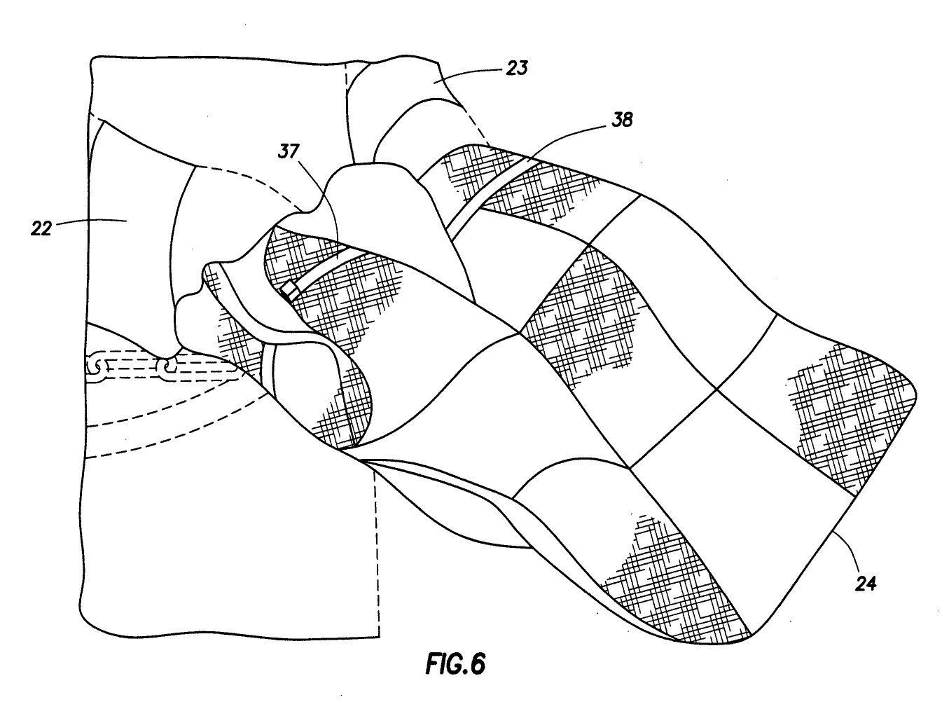

FIG. 6, in operation the hands of the prisoner are inserted into the invention 24, with the left hand in the left side compartment and the right hand in the right side compartment. The wrist straps 22 and 23 have a hook 35 and loop 36 velcro system attached. Wrist straps 22 and 23 are secured around the left and right wrist of the prisoner. The cap lock buckles 29 and 30 are interlocked causing the fabric body to form a bag around the hands of the prisoner. The plastic ties 37 and 38 are secured adjusting the bag around the hands of the prisoner preventing the use of the hands for grasping, or pulling.

The invention has been described in detail with particular reference to certain preferred embodiments thereof, but it will be understood that variations and modifications can be effected within the spirit and scope of the invention.

The embodiments of the invention in which are intended to be claimed are defined as follows:

1. An apparatus for restraining a prisoner's hands preventing the grasping or holding of an object comprising:
a fabric having front and back sides;
the fabric including upper and lower sections;
said upper section having an appendage attached at one end and a second appendage attached at the other end;
the upper section having a plurality of grommets inserted into said upper section;
a strap affixed to the upper section and a second strap affixed to the lower section; and
a cap lock buckle attached to said strap and another cap lock buckle attached to said second strap whereby when said cap lock buckles are in a locked position said fabric forms a compartment around said prisoner's hands.

2. The apparatus as recited in claim 1 whereby said appendages are designed to receive a wrist strap to wrap each wrist of the prisoner.

3. The apparatus as recited in claim 2 further comprising a string inserted in the plurality of grommets allowing the fabric to be adjusted around the prisoner's hands.

4. The apparatus as recited in claim 2 whereby said wrist strap includes a hook and loop fastening system.

5. The apparatus as recited in claim 1 further comprising webbing guides are attached to the front and back sides of the fabric.

6. A restraint device for securing a prisoner's hands comprising:
a fabric having front and back sides;
the fabric including upper and lower sections and said fabric joined together forming an inner pocket;
the inner pocket including left and right side compartments;
the upper section having a plurality of grommets inserted into said upper section;
a strap affixed to the upper section and a second strap affixed to the lower section; and
a cap lock buckle attached to said strap and another cap lock buckle attached to said second strap.

7. The apparatus as recited in claim 6 further comprising a plastic tie inserted in the plurality of grommets allowing the fabric to be adjusted around the prisoner's hands.

8. The apparatus as recited in claim 6 whereby said wrist strap includes a hook and loop fastening system.

9. The apparatus as recited in claim 8 further comprising webbing guides are attached to the front and back sides of the fabric.

10. A method of restraining the free movement of a prisoner's hands comprising the steps of:
a. restraining the hands of a prisoner at the wrists; and
b. inserting the hands of said prisoner inside a fabric, said fabric having front and back sides, the fabric including upper and lower sections and said fabric joined together to forming an inner pocket, the inner pocket including left and right side compartments; the upper section having a plurality of grommets inserted into said upper section, a strap affixed to the upper section and a second strap affixed to the lower section, and a cap lock buckle attached to said strap and another cap lock buckle attached to said second strap.

11. The apparatus as recited in claim 10 further comprising a plastic tie inserted in the plurality of grommets allowing the fabric to be adjusted around the prisoner's hands.

12. The apparatus as recited in claim 10 whereby said wrist strap includes a hook and loop fastening system.

13. The apparatus as recited in claim 12 further comprising webbing guides are attached to the front and back sides of the fabric.
